(19) United States
(12) Patent Application Publication
(10) Pub. No.: US 2025/0288277 A1
(43) Pub. Date: Sep. 18, 2025

(54) IMAGE-GUIDED ROBOTIC SYSTEM WITH REMOTE GUIDANCE, IMAGE STEERING, MULTI-PLANE IMAGING, NEEDLE VISUALIZATION ENHANCEMENT, AND CONSTRAINED BACKDRIVING

(71) Applicant: Mendaera, Inc., San Carlos, CA (US)

(72) Inventors: Alex Hassan, San Carlos, CA (US); Jason Wilson, San Carlos, CA (US); Ben Greer, San Carlos, CA (US); Andrew Robinson, San Carlos, CA (US); Yanan Huang, San Carlos, CA (US); Caitlin M. Romanczyk, San Carlos, CA (US); Polly Charlene Ma, San Carlos, CA (US); Josh Defonzo, San Carlos, CA (US)

(21) Appl. No.: 19/079,820

(22) Filed: Mar. 14, 2025

Related U.S. Application Data
(60) Provisional application No. 63/565,416, filed on Mar. 14, 2024.

(51) Int. Cl. A61B 8/00; A61B 34/00; A61B 34/30; A61B 34/35

(52) U.S. Cl. CPC A61B 8/4218; A61B 34/25; A61B 34/35; A61B 2034/305

(57) ABSTRACT

An interventional robotic device paired with an imaging device, for which a remote user selects targets or ‘waypoints’ on a remote interface and the robot subsequently maneuvers to satisfy commands implied by those targets or waypoints is described herein. By coupling a robotic targeting device to an ultrasound imager, the imaging plane is adjusted to match the measured position and orientation of the instrument tip, and/or the plane and robotic device adjusts to be optimally positioned and oriented relative to an anatomic target. By coupling a robotic targeting device to a multi-plane-capable ultrasound imager, the anticipated trajectory and/or intersection point of the instrument on/in the imaging planes may be displayed atop the ultrasound image. By coupling a robotic targeting device to an ultrasound system that has needle visualization enhancement features, the measured position and orientation of the instrument is used as inputs to the visualization features to most effectively enhance the instrument. Inverse kinematics (IK) based constrained backdriving allows the user to only focus on the part they need to control and assists the rest of the motion kinematically in a human-in-the-loop fashion.

### CROSS-REFERENCE TO RELATED APPLICATIONS

This application claims priority to U.S. Provisional Application No. 63/565,416 filed on Mar. 14, 2024, which is incorporated by reference herein in its entirety. This application also relies on U.S. Pat. No. 12,011,239, which is incorporated by reference herein in its entirety.

### FIELD OF INVENTION

This application is in the field of medical imaging systems.

### BACKGROUND

High-quality medical intervention may require sound clinical judgment, technical expertise, as well as timely and effective care delivery. However, delivering high-quality intervention at scale is challenging due to current methods of healthcare distribution. Healthcare providers with sufficient skill to perform an intervention are often far from the location where an intervention would most appropriately take place, resulting in complex coordination amongst departments and procedure locations within an institution, practitioners having to service multiple geographically disparate facilities, and/or patients having limited-to-no access to the intervention at all.

Specifically related to minimally invasive image-guided procedures, users must have spatial reasoning ability, as well as know how to effectively utilize imaging and minimally invasive devices. Despite the availability of affordable, non-radiating, real-time imaging such as ultrasound, its use is limited due to challenging interpretation. Few healthcare providers perform image-guided intervention at high-volume due to procedural complexity. This complexity can be associated with four fundamental challenges.

Image acquisition. Image acquisition may be challenging for healthcare providers due to various factors, including but not limited to poor motor skills, poor acoustic coupling, poor understanding of how to hold, apply, rotate, and angulate probe relative to the body to establish a relevant imaging window.

Image interpretation. Interpreting images (e.g., reading ultrasound images) may be challenging for providers due to various factors, including varying levels of understanding how ultrasound images are formed, how anatomy and pathology appears on ultrasound, and how to create cognitive three-dimensional (3D) information from a series of 2D planar images.

Intervention planning. Defining an optimal trajectory for an instrument (e.g., a needle) to hit an intended target with consistency, while avoiding anatomic constraints (e.g., avoiding contacting an unintended part of the anatomy) is challenging for providers due to various factors, including the absence of being able to consistently acquire, interpret 2D images, as well as derive a 3D volume from that information. Another challenge providers may face include the inability to contextualize the 2D images with respect to surrounding anatomy, which is more intuitive using 3D information.

Intervention execution. Even if an optimal trajectory for an instrument is defined, it is challenging for providers to consistently initiate and follow-through on their planned target due to various factors, including use of non-dominant hand to execute tasks, the ability to introduce instruments with the appropriate force and control with a single hand, challenge associated with obtaining optimal ultrasonic view of instrument tip while maintaining view of relevant anatomy, poor motor skills, steep needle insertion angles yielding poor reflection of sound waves, and planned versus actual needle path due to deflection. Moreover, manual techniques require the intervention plan to be created in the mind of the provider based on a mental map created from scanning across the anatomy. The intervention execution requires the provider to then manipulate a needle to through the skin to a target using a single image as a reference.

Overcoming these challenges requires substantial training and practice to estimate the optimal entry point, trajectory, and depth for an interventional instrument to reach a target that is being imaged by the US probe. This specialized skill is difficult to acquire and precludes many clinicians from performing successful ultrasound guided percutaneous interventions.

Solutions exist for more effectively distributing clinical judgment in certain limited fields of use. For example, tele-psychiatry, tele-primary care, and tele-radiology allow remote providers to connect with patients or exams and render clinical decisions. Other existing applications include tele-guidance in operating rooms, where physicians are able to connect via bidirectional video and audio to proctor and/or support cases with verbal or gestural instructions.

However, these existing virtual communication and collaboration solutions are limited to scenarios where there is no physical interaction. Remotely deploying physical capability or manual skill has not yet been demonstrated at scale in healthcare. This is largely due to significant technical requirements (hardware and software) for systems that would enable such remote interventional capabilities. One domain where this has been attempted is remote surgery, such as remote laparoscopic surgery with the Da Vinci surgical system, where a user tele-operates a robotic system that is in a different location than the control unit. However, this type of system, which relies on continuous, very low-latency roundtrip communication of video and telemetry data, is not deployable with modern internet infrastructure. This limitation will exist for any system where the user is expected to feel as though they are controlling the device in real-time.

In scenarios where a provider who intends to perform a minimally-invasive image-guided intervention has the expertise to interpret images, it is often useful to be able to visualize the instrument and anatomy in in an imaging plane that is not aligned directly with the long or short axes of the probe during the procedure.

A first example of this is a scenario in which the provider begins inserting a needle and it deflects out of the plane of the ultrasound image. The provider will not be able to maintain a view of the instrument and their originally identified anatomic target, because the instrument is deflecting away from that target. When this happens, the provider needs to determine how instrument is now aimed, so that they can decide if and how to make an adjustment. Adjustment typically involves retracting the instrument and starting the procedure again, which is clinically undesirable. Proceeding without adjustment typically requires the provider to move the imager back and forth between the target and the instrument, and mentally try to track the relative 3D positions of these, which both is a challenging maneuver and detracts from the value of image-guidance in general.

A second example is a procedure in which the provider is unable to obtain an optimal view of the target with the ultrasound beam at the start of the procedure, making it hard or impossible to perform the procedure with a view of the instrument and anatomy or plan an optimal instrument trajectory.

In ultrasound, very few products exist that allow for substantial adjustment of the plane of the ultrasound beam. For those ultrasound systems that have this capability, configuring the ultrasound beam is often so complex that it is not surfaced to the user as a controllable parameter. Even when it is controllable, a user struggling with the above challenges would not understand in what direction to maneuver this beam, since they are necessarily unaware of where the instrument has gone or what the optimal imaging plane is (in the two scenarios, respectively). These problems compound with other problems with ultrasound-guided procedures, including that the spatial awareness and hand-eye coordination required to (1) choose an optimal trajectory, (2) keep the instrument in that trajectory during insertion, and (3) know where the needle tip is, are challenging to master.

It can also be useful to visualize the instrument and anatomy in more than one imaging plane. A first example is during ultrasound-guided vascular access, where procedures are sometimes performed “in-plane”, where the long-axis of needle and long-axis of vessel are both in the ultrasound imaging plane, so that the needle can be wholly visualized as it approaches the vessel, and the needle tip can be visualized as it punctures through the top wall of the vessel. This approach, however, is unable to see how the needle is oriented relative to the cross-section of the vessel, and it is possible for the needle to be near the sidewall without the operator realizing (i.e. needle is not in the center of the cross-section). Some healthcare providers employ the “out-of-plane” technique, where the cross-section of the needle and vessel are in the ultrasound imaging plane. This technique has the inverse consequences; the centrality of the needle relative to the vessel is easy to determine, but the trajectory of the needle and the depth of the needle tip are comparatively harder to ascertain.

A second example is a biopsy, where a provider will typically use a single technique (in-plane or out-of-plane) to place the needle in the anatomic target of interest. It is beneficial to know from which region of the target the provider is going to take a sample, which would require seeing the position of the needle tip in both the in-plane and out-of-plane views.

Very few products exist that can deliver ultrasound images in multiple imaging planes concurrently. Using these types of ultrasound products can help in the clinical scenarios above, but operators also must deal with the problems of choosing and maintaining a trajectory, and knowing where the needle tip is during insertion. When there are multiple imaging planes being displayed, errors in any of these tasks lead to even more confusion than when the operator is using only one imaging plane.

Related to the third problem (knowing where the needle tip is during insertion), some commercially available ultrasound devices have a feature designed to enhance the echogenicity of needles during their insertion through tissue. Poor needle echogenicity can be due to multiple factors, including that sound waves reflect of the needle surface and away from the ultrasound transducer. Needle visualization enhancement features often leverage a combination of (1) adjusting the reception and transmission of the ultrasound waves so that they are oriented towards the expected location and angle of the needle and (2) using post-processing image adjustments to enhance and highlight linear structures (such as a needle) in the resultant ultrasound image. Both methods require the ultrasound system to assume information about the position and orientation of the needle relative to the transducer. In commercially available systems, this is often handled by requiring the user to input the expected needle angle on a user interface. However, due to the problems cited above, it is hard for a user to identify and maintain an optimal angle of approach when using a freehand technique, making it hard to precisely know what approach angle to input into the ultrasound system.

Devices exist to help providers overcome some of these above challenges, such as robotic systems that help the user acquire an image, identify anatomy, plan an approach, and execute the procedure. These devices can have varying levels of automation and robotic control, from only visual guidance to complete automation of the procedure. An intermediate operating mode is backdriving, where a user manually and collaboratively adjusts the needle trajectory or interacts with the robot in a haptic manner. Direct backdriving can be used to move the robot into set-up configurations or to quickly establish an initial position, and then the user may switch to a graphical user interface interaction for various targeting modes, such as tapping and dragging on the interface themselves, using automatically segmented pixels to automatically aim, or by leveraging the guidance of a remote provider, all of which can enable fine, precise aiming of the robotic device.

In many cases, the user may still want to manually interact with the arm for fine targeting, due to the more natural workflow/usability, and/or enhanced sensory interaction, such as haptic/tactile feedback. Unconstrained backdriving may not adequately service the user need of desiring fine targeting capabilities associated with automated motion while preserving the direct user interaction associated with backdriving.

### SUMMARY

An interventional robotic device paired with an imaging device, for which a remote user may select targets or ‘waypoints’ on a remote interface, and the robot will subsequently maneuver to satisfy the commands implied by those targets or waypoints may be described herein.

A combination of an adjustable ultrasound imaging plane and robotic guidance technology may be described herein. Robotic and non-robotic ultrasound guidance systems have primarily focused on keeping an instrument in either the long- or short-imaging axis and perpendicular to the transducer, rather than using a non-primary or non-perpendicular plane or adjusting the imaging plane during the procedure. By coupling a robotic targeting device to an ultrasound imager that can adjust the imaging plane, the imaging plane can be adjusted to match the measured position and orientation of the instrument tip, and/or the plane and robotic device can adjust to be optimally positioned and oriented relative to an anatomic target.

The combination of multi-plane ultrasound imaging and robotic guidance technology may be described herein. Robotic and non-robotic ultrasound guidance systems have primarily focused on keeping an instrument in a single ultrasound imaging plane, rather than using multiple concurrent imaging planes to visualize an anticipated trajectory and/or establish that trajectory. By coupling a robotic targeting device to a multi-plane-capable ultrasound imager, the anticipated trajectory and/or intersection point of the instrument on/in the imaging planes may be displayed atop the ultrasound image.

A combination of needle visualization enhancement features and robotic guidance technology may be described herein. Existing needle visualization enhancement features rely on the ultrasound system assuming a position and orientation of the needle as it enters the body. By coupling a robotic targeting device to an ultrasound system that has needle visualization enhancement features, the measured position and orientation of the instrument can be used as inputs to the visualization features to most effectively enhance the instrument.

Inverse kinematics (IK) based constrained backdriving lets the user only focus on the part they need to control and assists the rest of the motion kinematically in a human-in-the-loop fashion may be described herein. Given the same framework, it may be very flexible and easy to implement a range of constrained backdriving modes to meet specific desires, and/or migrate to different robot designs. The user experience mainly depends on the motion constraint and user touchpoint selections for a given robot.

A robotic system with software-controlled angular adjustment may be described herein. A user interface may be used to initiate a software command that moves joints of a robotic device to change the angle of the robot while maintaining one or more fixed constraints such as a target location or end-effector height.

### BRIEF DESCRIPTION OF THE DRAWINGS

FIG. 1 is an example of a user selecting a target on a remote interface;

FIG. 2 is an example of a catheter-based robotic system;

FIG. 3 is an example of remote center of motion (RCM) constraint;

FIG. 4 is an example of a system converting guidance or commands shown on the external view to similar guidance or commands in a frame of the imager;

FIG. 5 is an example block diagram of a communication architecture to facilitate remote support of the robotic system;

FIG. 6 is an example ultrasound system with an adjustable imaging plane

FIG. 7 is an example of a robot mounted to an ultrasound probe;

FIG. 8 is an example of the instrument being further inserted into a patient;

FIG. 9 is an example of an ultrasound imager steering a beam to acquire multiple views of an anatomy;

FIG. 10 is an example of multiplane robotic targeting with ultrasound;

FIG. 11 is an example of needle visualization enhancement without and with a robot;

FIG. 12 is an example flowchart of the constrained backdriving framework;

FIG. 13 is an example of a 5-DoF robot design;

FIG. 14 is an example of a 3-DoF robot design;

FIGS. 15a and 15b illustrate an example of RCM backdriving;

FIGS. 16a and 16b illustrate an example of PnP backdriving; and

FIG. 17 is an example of a patient side robotic system for percutaneous instrument intervention.

### DETAILED DESCRIPTION OF THE DRAWINGS

An image-guided robotic intervention system (“IGRIS”) may be comprised of a robotic instrument guide, an Imaging Device, a sterile barrier, a patient side computer with a user interface and system software including a user interface. Each of these may be employed by healthcare professionals (i.e. users) when performing one or more medical procedures on patients. The IGRIS relies on imaging to provide the user with a real-time view or imaging of patient anatomy and pathology, as well as an intended target or targets for the procedures, software that allows a user to plan an approach or trajectory path using either the real-time imaging or the robotic device. The software may allow a user to convert a series of 2D images into a 3D volume, and localization sensors to determine the pose of the devices relative to the patient, patient anatomy and pathology.

The IGRIS may be comprised of the following hardware components: (1) an imaging device capable of imaging objects, such as patient anatomy and pathology, (2) a computer to perform calculations and algorithms based on those images, (3) a robotic arm (e.g., a robotic manipulator and an instrument guide) that affects the use of an instrument which interacts with the patient anatomy and pathology, and (4) external sensors (e.g., IMU and cameras). Here, the Health Care Provider (“HCP”) or user interacts with the system and potentially the instrument itself to execute the procedure.

The imaging may be real-time ultrasound; the instrument is intended to be introduced percutaneously, directed in an approximately straight line to an anatomical target. The imaging, a computer, and robotic arm may be handheld. In some embodiments, computer processor operations, including and not limited to machine learning and/or artificial intelligence, may be offloaded to the cloud for processing.

Alternatively, the elements may be mounted on at least one positioning apparatus where the elements are manipulated manually or via robotic actuation.

As such the HCP interacts with IGRIS to physically stabilize the system and acoustically couple the ultrasound transducer to the patient; acquire and interpret ultrasound images; configure, plan and execute an intervention using typical computer input interfaces (e.g., touch screen, mouse, keyboard, joy stick, track ball, etc.); and may collaboratively manipulate the instrument to optimize the intervention.

IGRIS may not include an Imaging Device and may be operated to couple with existing Imaging Devices. For example, IGRIS may be attached to a standard ultrasound device and operate in the same manner as an IGRIS with an integrated Imaging Device. This may be represented at a “modular robotic arm”.

The modular robotic arm may be used in a wide variety of percutaneous procedures on an object (e.g., human anatomy). In some embodiments, a user may hold the imaging device (e.g., ultrasound probe) with the modular robotic arm mounted in one hand, the modular robotic arm points an instrument guide at the target, and the user manually inserts the needle through the instrument guide. In some embodiments, the instrument guide may be removable and replaceable. Preferably, the modular robot arm may operate in a sufficiently large workspace to accommodate inserting a needle near the ultrasound probe, as well as far away to enable a wide variety of percutaneous procedures. Further, the instrument guide and the robotic joints of the modular robotic arm are slim in profile to avoid collision with the imaging device, the robot itself (self collisions), and surrounding external anatomy. The modular robotic arm may also comprise gripping and insertion mechanisms to allow the modular robotic arm to measure and/or control the instrument insertion degree of freedom. Modular robotic arm (1) may be universally mountable to a plurality of Imaging Devices, (2) may be mountable around an existing cabling (which may not be removable) in a plurality of mounting positions (3) may include a homogenous kinematic architecture, and (4) may include low-profile wrist via remote actuators (e.g. cable drives).

The modular robot arm may support backdriving, which allows the user to adjust the instrument guide manually. This backdriven motion may be constrained or unconstrained to help the user more easily adjust the needle trajectory. For example, the instrument guide (e.g., needle guide) may point at the target while allowing the user to adjust the entry site—or vice versa. For the backdriving mode to be intuitive, the robot may include homogeneous kinematic properties throughout the workspace keeping it away from singularities.

In some embodiments, the modular robot arm may be symmetrically mounted on an Imaging Device to ensure flexibility in probe/robot orientation, thereby facilitating left- and right-hand use, as well as robot in front and behind configurations. This may also facilitate in plane and out of plane clinical procedures.

The modular robotic arm may be coupled to a computer. The computer is calibrated such that defining anatomy in the image space (pixels) allows the robot to point the end effector to target the same anatomy in physical space

The modular robotic arm may include a first joint that can fit around existing Imaging Device cabling (e.g., a fixed ultrasound probe cable), yet still rotate freely. In other embodiments the mount may not rotate freely in favor of allowing for the robot to rigidly attach to the probe in a plurality of fixed rotation angles. The most common angles are for cardinal locations that facilitate right hand in plane, left hand in plane, behind image out of plane and in front of image out of plane. The modular robotic arm may attach to the Imaging Device via a semi permanently mounted Imaging Device specific receptacle. The receptacle/robot interface may be standardized such that the robot may attach to any Imaging Device with a corresponding receptacle.

Modular robotic assembly may mount to any Imaging Device (e.g., any ultrasound probe) using an interface. A specific interface may exist for every compatible Imaging Device. One end of the interface may include a unique design to couple to the external housing of Imaging Device. The other end of the interface may include coupling geometry to attach to the modular robotic arm. The coupling geometry may include a number of features: (1) keying geometry to control the orientation of the mechanical mounting of the robot to the Imaging Device; (2) locking features so that the modular robotic assembly may be locked on top of the Imaging Device; and (3) electrical contacts to provide power and communication to accessories that may be embedded in the interface including buttons, cameras, and an IMU.

In some embodiments, modular robotic assembly comprises complimentary coupling geometry that attaches to the interface at the first rotational joint. In some embodiments, the robotic arm comprises a relief that allows the joint to slip past cabling that drives the Imaging Device. For example, this relief may create a distinctive C shaped geometry as opposed to the traditional O shaped geometry of a traditional robotic joint. In some embodiments, the components of the joint (e.g. bearings, bushing, etc.) must “jump the gap” to provide stiffness through large rotations. In some embodiments, a recirculating bearing may be used. In some embodiments, when paired with multiple drive gears, the joint can still rotate 360 degrees around the Imaging Device. In some embodiments, when paired with a single drive gear, the joint may allow almost 360-degree rotation.

Aspects of the present invention may operate using many kinematic geometries as understood by one of skill in the art, including and not limited to parallel manipulator systems. In some embodiments, the first rotational joint may be mounted such that the rotational axis is along the axis of the probe, acting similar to a turret. In some embodiments, the turret may position the rest of the modular robotic arm symmetrically around the Imaging Device. This enables the modular robotic arm to be positioned arbitrarily and symmetrically around the Imaging Device in rotation and in a mirroring fashion.

The most general kinematic architecture is 6 degrees of freedom (DoF), allowing for the instrument to be positioned arbitrarily with respect to the imager, including arbitrary position and orientation of the instrument. A particularly useful kinematic architecture is 5 DoF, where the sixth degree of freedom controlling the rotation around the instrument is not controlled. This is particularly useful where the robot is intended to guide a percutaneous instrument where the rotation about the instrument—or needle—is constrained or left up to the user. In this configuration, the trajectory the needle takes along its axis of symmetry is completely controlled by the robot, and the needle rotation is not.

A simpler version is a 3 DoF robot, where the robot is able to orient the instrument or needle in a plane. This includes 2 DoF for position and one angle for orientation. This configuration allows the user to set the skin entry location, and the angle of entry for the instrument to hit the correct target. This is particularly useful when the imager is an ultrasound, where imaging is a cross section of anatomy. Aligning the robot to have the plane of control coincident with the ultrasound image is most clinically relevant. Similarly, it is also clinically relevant to have the plane orthogonal to the imaging plane for out of plane procedures.

The extreme case where the robot is a single degree of freedom is also clinically relevant. In an embodiment, the entry site relative to the ultrasound probe may be fixed, and the robot is only controlling the angle of instrument insertion.

Modular robotic arm comprises corresponding motors or actuators for the rotational joint. In some embodiments, the motors may be housed in the joint link just proximal to joint of interest. The motors or actuators may be coupled to the joint rotation or translation using a mechanical drive train like gearing or cables and capstans.

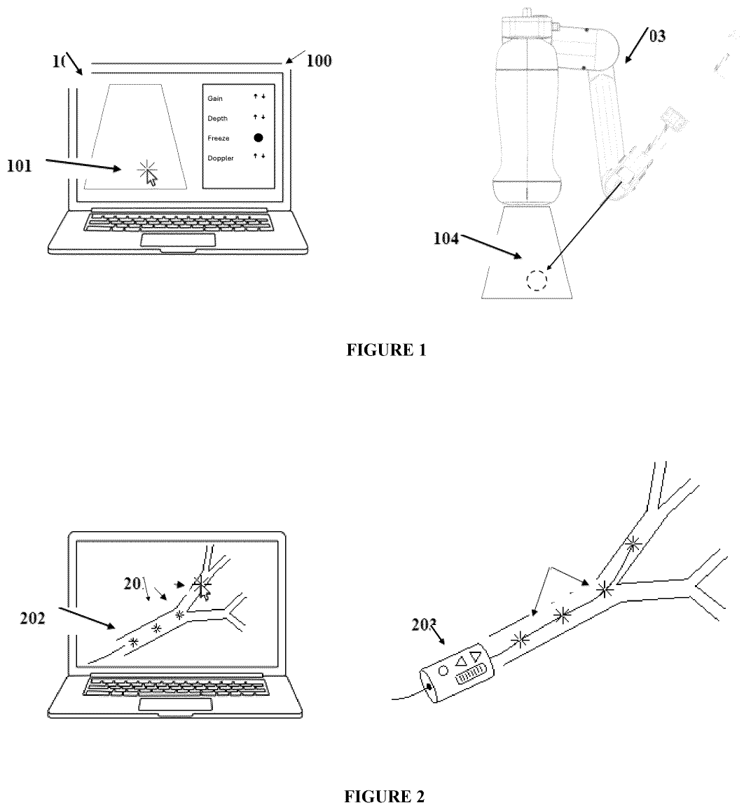

The system may include the capability to control the robotic system remotely. FIG. 1 shows an example of a user selecting a target 101 on a remote user interface 102, such as laptop 100, and the robotic system 103 physically aiming at the corresponding location in the ultrasound image 104. In this embodiment, the remote user interface is an duplicate of the robotic system user interface that is patient side (not shown).

The act of selecting a pixel location in an ultrasound image to command the robot to point an instrument at a target is referred to as tap-to-target (T2T). Dragging a finger on the user interface to adjust the instrument angle is referred to as drag-to-rotate (D2R), and is further explained below under the general category of “software-controlled angular adjustment”. These are fundamental user interactions in a graphical user interface to command the robot to guide the instrument to a target.

The proposed system includes an interventional robotic device paired with an imaging device, for which a remote user may select targets or ‘waypoints’ on a remote interface, and the robot will subsequently maneuver to satisfy the commands implied by those targets or waypoints.

The approach of using “waypoint-style” commands in an interventional system is different than the tele-operation described in the background for traditional surgery systems, in part due to the less stringent latency requirements and obviation of need for fast roundtrip video and telemetry data. The robot motion is quasi-static (rather than continuous), and thus does not require a real-time closed loop or human in the loop with the remote user.

In addition to the benefits of solving the problems described above, a system like this can enable training with a remote trainer or proctor supervising and guiding the in-person user, or remote assistance in situations where in-person assistance is unavailable (for example, don't need a costly second person in the room to interact with the system, change settings, associate images with a patient record, etc., if someone is able to do control those functions remotely).

This may include a remote user tapping on the ultrasound image on a mirrored user interface, and having the robot automatically aim at the target, similar to the tap-to-target (“T2T”) function for an in-person user of the robotic system. A remote user may choose multiple waypoints, and the system may progressively satisfy them. For example, a remote user may select several biopsy targets (waypoints), to which the robotic device may move in a step-by-step fashion. The remote setting of waypoints can be used for a variety of types of robotic devices, such as orthopedic robots for spine surgery (where the remote user may select waypoints and angles of approach associated with pedicle screw placement, atop one or more fluoroscopic images), or laparoscopic surgical robots (where the remote user may select waypoints for the robotic arms to achieve during setup so that they are positioned properly relative to the patient and resultant cannula locations, atop a live camera image). This approach to waypoint-style control may be applied to different types of robotic systems. FIG. 2 is an example of a catheter-based robotic system, where a remote user may tap several waypoints 201 along an airway 202 (or other lumen such as the GI tract or a vessel), using a “birds-eye” view, and the system may then advance the catheter 203 automatically by advancing to each waypoint 201 in succession.

In one embodiment, where a remote user has control of the entire mirrored user interface, the remote user may interact with the UI just like the in-person user. This may include allowing them to: (i) Activate other robotic functions such as software-controlled angular adjustment; (ii) Control ultrasound functions like gain, depth, freeze, and preset; (iii) Save images or video media; (iv) Annotate or measure atop the ultrasound image; and (v) Configure system settings.

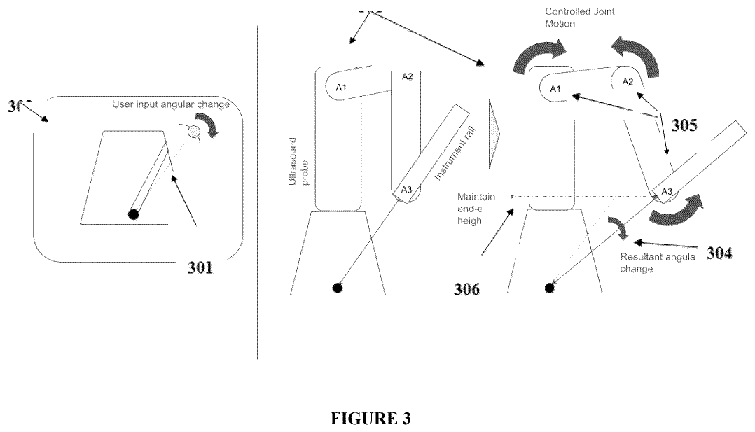

Software-controlled angular adjustment involves the use of software and robotic joints to satisfy a user-requested adjustment to the angle of the robot while maintaining a target location. The robot in this case is controlled using a remote center of motion (RCM) constraint. FIG. 3 an example of remote center of motion (RCM) constraint. The user inputs a desired angular change 301 on the user interface, with a defined target location on the medical image 302. This is referred to as drag-to-rotate (D2R). The system sends commands to the robot 303 (in this case, a three degree-of-freedom planar robot with joints A1, A2, and A3) to change the instrument angle 304 while remaining aimed at the target. In this robot setup 303, there are three degrees of freedom 305, for controlled joint motion, (for example, points A1, A2, and A3 in FIG. 3) for simply changing the angle and maintaining the target location. FIG. 3 shows how the degrees of freedom 305 are used to abide by the additional kinematic constraint of maintaining the height of the end effector 306.

The system may optionally include an external view of the in-person operator, procedural area, patient, and physical system, such as via an overhead camera, as well as display video of the remote operator. The system may also include one- or two-way audio communication with the remote operator. The remote user may also use telestration (remote drawing overlays atop the user interface or the external view) to provide guidance to the in-person operator. The remote user may optionally have control of the external view, either by directly controlling the camera or by controlling the resulting image (for example, digital zoom).

The remote operator may interact with multiple types of interfaces, including a web app, a smartphone, tablet, or their own instance of the physical user interface, such as their own cart and touchscreen. The remote interface may include a digitized visualization of the robotic system so the external user has visual context of the robot pose at any given time.

The remote interface may be used for support or service operations related to the device. For example, during a procedure, support or service personnel may leverage audio, video, sharing of user interface elements, or sharing of medical images, to troubleshoot or triage issues. They may also leverage the ability to communicate using these same mechanisms to ask the in-person user to test or try various operations with the system. In embodiments where the remote user has the ability to modify the system software remotely, they can use this capability to run system diagnostics, troubleshoot issues, and otherwise provide guidance to the user. Modifying the software may include changing configuration parameters, updating software modules, or remotely installing a new disk image inclusive of the operating system and robotic software application.

The remote interface may have a portal to connect to historical or real-time system data, such as robot telemetry data, system health, faults and issues, and utilization. This system data can be used concurrently with the live audio and video capabilities to more effectively provide remote assistance or troubleshooting to the primary user. The portal may automatically associate the live audio or video call with the historical or real-time data associated with the current system, using identifiers such as the device ID, or with the current user, using identifiers such as the provider name or ID.

For robotic devices where the advancement, retraction, and/or activation of an attached instrument may be initiated remotely, the remote user can initiate those functions. This may include activating instrument insertion by causing the robotic arm to move towards the patient. In alternate embodiments, the remote user may trigger an actuator to physically drive the needle forward or backward until it reaches the pre-selected target.

In yet another alternate embodiment, the remote user may actuate instrument functions when the instrument reaches the target. This includes but is not limited to heat, radio frequency (RF) energy, injection, aspiration, ablation, biopsy, mechanical tissue interactions (gripping, scissors), high intensity ultrasound and the like.

In other embodiments, the remote user may control the imager itself. This may include remotely steering the ultrasound beam by requesting different transducer parameters. Alternatively, if the ultrasound probe is attached to a robotic arm, the remote user may trigger motion of the ultrasound probe in any of the six degrees of freedom (three positional, three rotational). Another example may include the in-person user holding the end effector of the robot, and then the software controlling the position of the imager by running the kinematic solver “in reverse” (i.e. treating the probe as the end effector).

The system may contain an approval workflow, where the remote user or the in-person user may propose targets/waypoints that have to be approved by the other. This may be an optional feature that may improve the safety of the remote command (by requiring the in-person user to approve it before the system moved), reduce the burden on the remote user (by allowing the in-person user to propose a target), or both. The approval workflow may be designed to be uni- or bi-directional; either side can propose targets, and either side can approve targets. For systems where there is onboard AI to assist with target/waypoint/path identification, the approval pipeline may allow either the in-person or remote-user to approve AI-suggested waypoints.

The approval may either be a one-time selection or a continuous selection (e.g. dead-man's switch). Two example approval workflows are:
a. Workflow A
   i. Remote user selects target atop ultrasound image on their user interface;
   ii. Virtual target and confirmation button appear on the in-person user interface;
   iii. In-person user taps confirmation button;
   iv. Robot moves to achieve selected position;
b. Workflow B
   i. Remote user selects target atop ultrasound image on their user interface;
   ii. Virtual target and confirmation button appear on the in-person user interface;
   iii. In-person user presses-and-holds confirmation button (dead man's switch);
   iv. Robot moves to achieve selected position while button is held;
   v. Robot stops motion if user releases confirmation button.

Since the system may be used in a collaborative way with an in-person user and a remote user, it is likely that the system will have mechanisms for handling when in-person and remote commands disagree. For example, target or waypoint rejection may be implemented:
a. Latency-/time-based: if the latency is too high (e.g. slow internet speeds);
b. Image-based: if the image is sufficiently different from the image that was shown when the target was selected (e.g. in-person user moved imager); and
c. Position-based: if the robot/end effective is detected to be in a different place (e.g. in-person user backdrove the robot, or needle was inserted/retracted).
   Conflicts may be handled via an approval system as described above.

The system may allow the in-person user to request for a remote user to assist with their procedure. This can be done by the in-person request being placed in a queue, and the first-available remote user is then assigned to and connected to the in-person user. The system may also automatically suggest when it may be useful to bring in a remote user based on which procedure is being performed, sensed measurements from the robot or probe, or user interactions on the user interface.

The remote user may trigger media exports to PACS or other media repositories, similar to an in-person user, but may also initiate media exports that are sent to them (the remote user). An example may be the remote user triggering a DICOM image to be sent to them, to subsequently be used for billing.

In a more advanced embodiment, the system may convert guidance or commands shown on the external view to similar guidance or commands in the frame of the imager or the image itself. For example, the remote user may draw an “upward” arrow in the external view, which may correspond with a motion of the imager to the “right” from the in-person user's perspective; as long as the system understands the orientation of the imager relative to the external view camera, the software may be able to display guidance telling the user to move the imager to the “right”. This embodiment leverages information about the pose of the medical imager relative to the external view, for example, using optical tracking methods or information from an accelerometer. Using one or more cameras, which may or may not include the camera generating the external view, it is possible to determine the orientation of the medical imager relative to the external view.

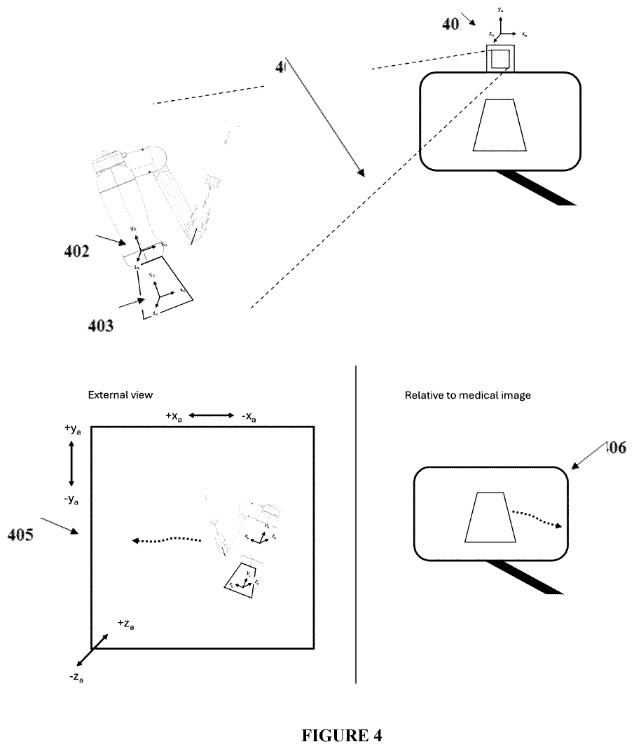

FIG. 4 is an example of a system converting guidance or commands shown on the external view to similar guidance or commands in a frame of the imager. In a third-person view, the external camera and its coordinate frame 401 (denoted by subscript ‘a’), the medical imager and its coordinate frame 402 (denoted by subscript ‘b’), and the medical image and its coordinate frame 403 (denoted by subscript ‘c’) are shown. The dashed lines 404 indicate the view of the external camera. Once the optical tracking and accelerometer data from 401 and 402 are used to identify the transformation from coordinate frame ‘a’ to coordinate frame ‘c’, it is possible to convert an on-screen drawing in the external view 405, such as the dotted line in the +xₐ direction, to a similar instruction shown relative to the medical image 406, which is in the +x_{c} and −y_{c} direction.

Additionally, or alternatively, the remote user may select targets in the external view 405, which are in-turn converted to targets or waypoints for the robot. For example, the user may select a desired skin entry site on the external view 405; as long as the system understands the position and orientation of the robot relative to the external view camera, the robot may move to satisfy that target. This embodiment may likely require the use of heuristics for determining the intention of the remote user, since it is hard to select a precise, 3D target using a 2D view (such as an external camera), and yet this 3D information is needed to perform a similar transformation as the above description that includes position in addition to orientation information. These heuristics could include, for example, template matching with calculations based on relative size; if the size of the medical imager or robotic device is known, then the anticipated size at various distances from the camera can be a priori calculated, and based on its relative size in the camera image, an estimated distance from the camera can be calculated. More advanced optical tracking (with optical tracking sensors and fiducials) may also be used for this purpose.

The term “remote” is broad and implies a range of distances between the remote user and the in-person user. This may include over the internet (e.g. in-person user is in California and remote user is in New York), on a local network (e.g. both users are in the same hospital, but in-person user is in a different area than the remote user), or via another wireless modality (e.g. user is in an adjacent control room, connected via Bluetooth).

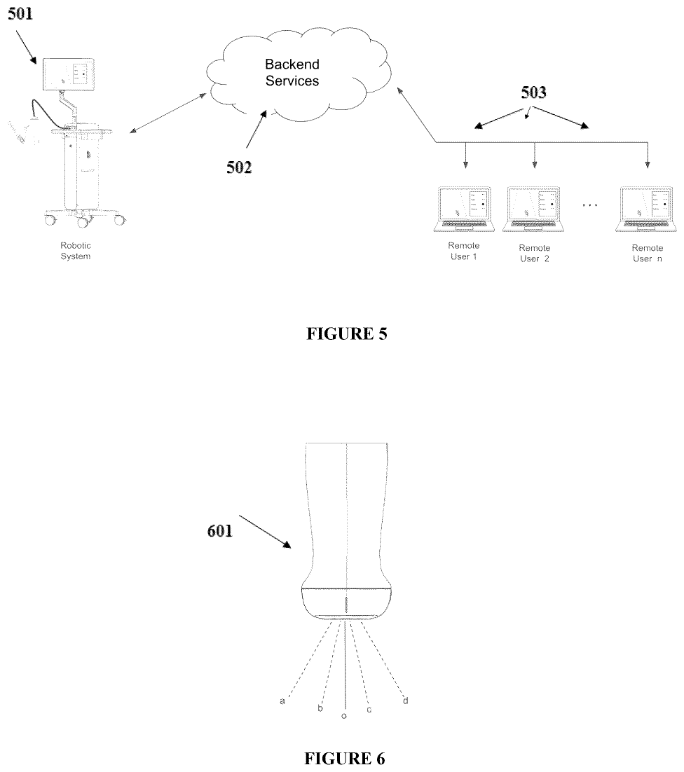

FIG. 5 is an example block diagram of a communication architecture to facilitate remote support of the robotic system. The robotic system 501 connects to backend services 502 that coordinate connection with the robotic system 501 and remote users 503. This includes establishing a secure connection between the robotic system 501, backend services 502, and remote users 503. Multiple remote users 503 may connect at once, requiring the backend services 502 to permission and coordinate user capabilities. For example, it may be required to ensure only one of the remote users 503 control the robotic system 501 at a time. This avoids race conditions with conflicting commands from multiple remote users 503. Simultaneous annotation may be enabled to allow multiple clinicians to collaborate on a case. Real time ultrasound imaging must also be provided to ensure that the remote users 503 can perform tasks that require clinical judgement based on current ultrasound scans. The backend services 502 may provide one way, or two-way video and audio communication to allow for a seamless conferencing experience between the health care provider at the robotic system 501 side and remote users 503. Finally, the backend services 502 may generate and maintain updated software configuration parameters, software module updates, application updates, driver updates, robot firmware, or disk images inclusive of operating system and all other software/firmware which may be deployed remotely to the robotic system 501.

A combination of an adjustable ultrasound imaging plane and robotic guidance technology may be described herein. By coupling a robotic targeting device to an ultrasound imager that can adjust the imaging plane, the imaging plane can be optimized based on the instrument pose, and/or the plane and robotic device can adjust to be optimally positioned and oriented relative to an anatomic target.

FIG. 6 is an example ultrasound system with an adjustable imaging plane. A typical imaging plane associated with imaging that is centered on the probe 601 (as with typical ultrasound probes) is denoted by letter ‘o’. However, certain probe technologies may allow the imaging plane to be steered left (e.g. ‘a’, ‘b’) and right (e.g. ‘c’, ‘d’). These technologies include phased arrays and matrixed transducer arrays. The drawing shows this steering as changes in angle of the beam, but the steering may also be performed as a lateral shift. For an ultrasound imager with multiple imaging beams, this steering may be done with one or more of those beams.

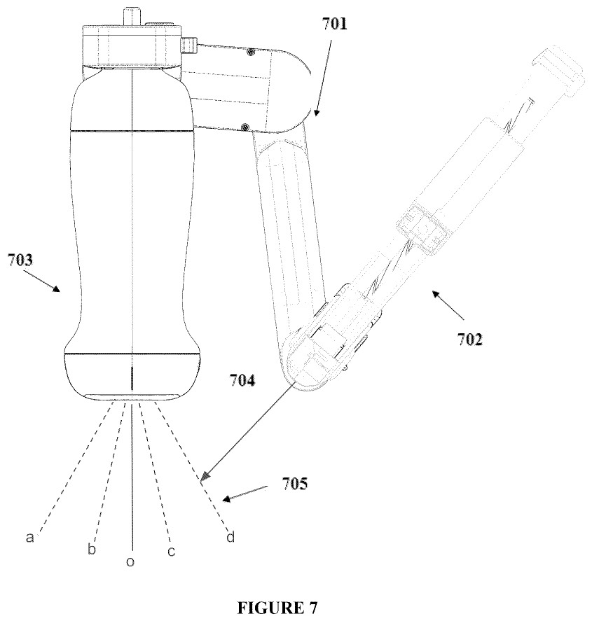

FIG. 7 is an example of a robot mounted to an ultrasound probe. When coupled with the robotic targeting device 701 that controls or measures the position of the instrument 702, the ultrasound transducer 703 may actuate arrays in such a way that the imaging plane follows the instrument tip 704 (or other location of interest) as the instrument moves through tissue. When the instrument is initially inserted, a cross section of the instrument tip 704 is imaged by image plane ‘d’ 705.

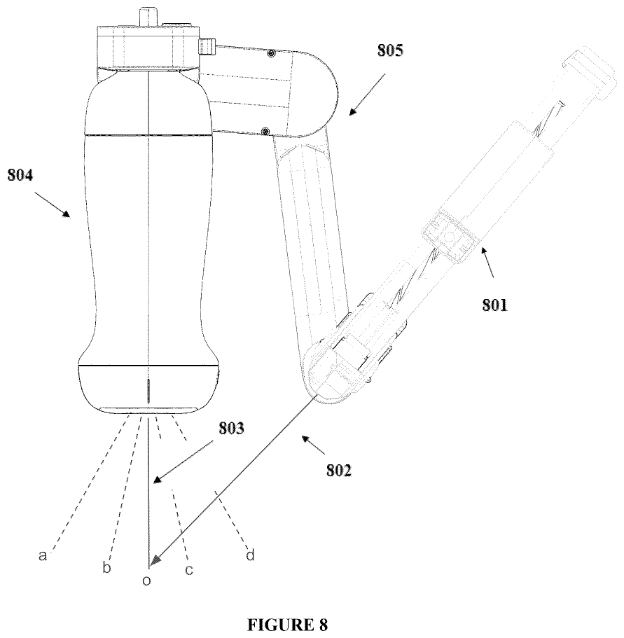

FIG. 8 is an example of the instrument being further inserted into a patient. As the instrument 801 is further inserted a cross section of the instrument tip 802 is imaged by image plane ‘o’ 803. In this way, the ultrasound image may follow the needle to the target, maintaining a cross section of the instrument tip 802 and the surrounding anatomy.

This closed loop tracking of the instrument tip 802 by the ultrasound imager 804 is uniquely achieved because the robot 805 is able to accurately control and measure the pose of the instrument 801 relative to the probe. The robot 805 may then transmit commands to the ultrasound probe 804 to image the correct plane to show the view of the instrument that is desired by the user.

Although, an imaging plane that is fixed aside from left to right motion, as illustrated in FIGS. 6-8, this concept may be extended to the general case where the robot is holding the needle at any pose (position and rotation) relative to the probe. If the ultrasound imager is sophisticated enough to provide images at any angle relative to the transducer, closed loop control may be used to image in planes that always show the needle contained in the image plane, and the cross section of the tip (or other significant location on the instrument simultaneously). In other words, steerable ultrasound imaging may be extended to the six degree of freedom instrument pose case, where the imager has enough degrees of freedom to steer the image planes to the instrument at any pose.

For example, a user may place the system on a patient as shown in FIG. 7 and perform an initial scan with image plane ‘o’. As they insert the needle, the robotic guide may detect that the needle has been inserted to the image plane represented by ‘d’. The system may update ultrasound plane to ‘d’, either automatically or in response to user input. As the user continues to insert the needle (thus causing it to intersect ‘c’ and ‘o’), the system may continuously or discretely update the ultrasound imaging plane accordingly. As described above, for a sufficiently sophisticated imager with multiple degrees of freedom, the imaging plane could be steered to match any six degree of freedom position of the needle tip.

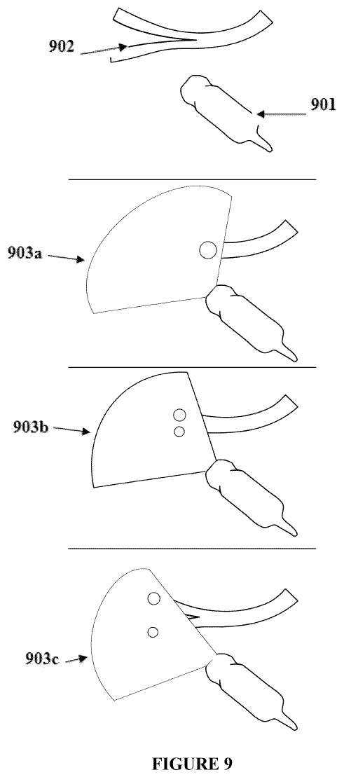

An imager with steerable ultrasound beam can be configured to collect multiple views of an anatomy and determine which is the optimal view for the intervention. FIG. 9 shows an ultrasound imager 901 sweeping through an anatomy 902 to acquire several views 903a, 903b, and 903c. Once an optimal view is identified, either automatically, by the user on the user interface, or by the remote user, because the position and orientation of the ultrasound beam relative to the probe is known, an attached robot with sufficient degrees of freedom can be configured to aim within that imaging beam. If it does not have sufficient degrees of freedom, the user can be provider with guidance on the user interface to encourage them to move the imager so that the optimal view is aligned with a pose that can be satisfied by the robot.

The combination of multi-plane ultrasound imaging and robotic guidance technology may be described herein. Multi-plane ultrasound imaging is very uncommon in the ultrasound market. Robotic and non-robotic ultrasound guidance systems have primarily focused on keeping an instrument in a single ultrasound imaging plane, rather than using multiple concurrent imaging planes to visualize an anticipated trajectory and/or establish that trajectory. By coupling a robotic targeting device to a multi-plane-capable ultrasound imager, the anticipated trajectory and/or intersection point of the instrument on/in the imaging planes may be displayed atop the ultrasound image. Furthermore, a user may select a desired trajectory and/or intersection point on one or more of the ultrasound images, and the robotic targeting device may move to satisfy that input. Multiple views may be made available simultaneously, and all trajectory selection modalities associated with those images may be made available simultaneously as well.

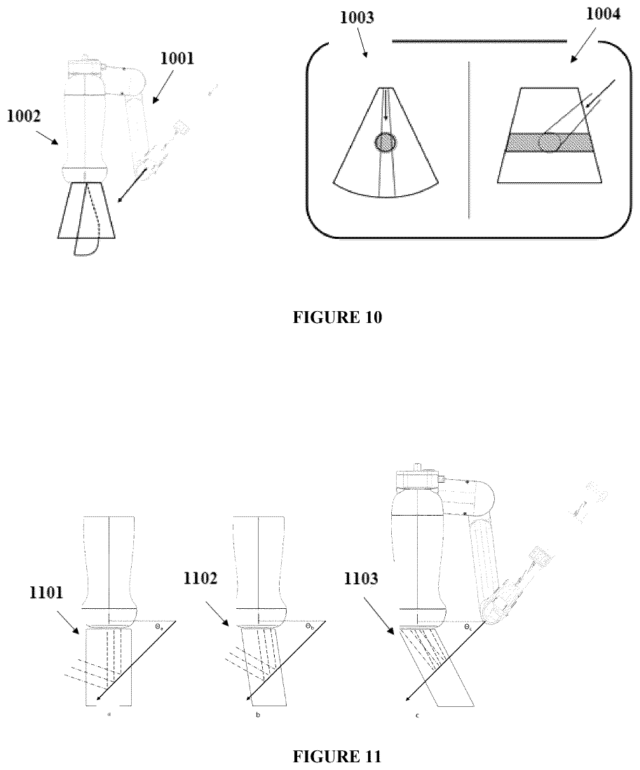

FIG. 10 is an example of multiplane robotic targeting with ultrasound. A robotic targeting device 1001 may be coupled to a multi-plane ultrasound probe 1002 with one or more adjustable imaging planes. This may result in a combination of the adjustable plane and multi-plane capabilities described above. Multi-plane ultrasound imaging may help less experienced users perform ultrasound guided procedures because they may visualize the needle and anatomy concurrently in multiple imaging planes and leverage the benefits of robotic technology to overcome the planning and hand-eye coordination challenges described above.

In FIG. 10 the user is accessing a vessel which is imaged cross-sectionally on the left 1003, and longitudinally on the right 1004. To completely define the desired location of the needle tip in 3D space, the user would need to define the target in both views 1003 and 1004. The longitudinal view (right) 1004 is required to define the desired position of the needle tip along the length of the vessel. The cross section (left) 1003 is required to define how centered the needle in the vessel. Either image can be used to define the depth to which the needle is inserted. The user may sequentially define those parameters by starting with the longitudinal or cross-sectional views.

A combination of needle visualization enhancement features and robotic guidance technology may be described herein. By coupling a robotic targeting device to an ultrasound system that has needle visualization enhancement features, the measured position and orientation of the instrument can be used as inputs to the visualization features to most effectively enhance the instrument.

FIG. 11 is an example of how coupling robotic guidance to an imager capable of needle visualization enhancement can improve the performance of the enhancement in a way that cannot be enabled without the robotic technology. View ‘a’ 1101 shows how needle echogenicity can be poor during ultrasound imaging, because the emitted sound waves reflect off the instrument and do not return to the transducer, resulting in poor echogenicity of the needle. View ‘b’ 1102 shows one version of needle visualization enhancement, in which the beam is steered toward the instrument, resulting in an improvement in signal return (shown as the return echoes being closer to the transducer in ‘b’ than in ‘a’), but the beam alignment is not optimal. This is because in views ‘a’ and ‘b’, the angle between the needle and the transducer, represented by theta-a (Qₐ) and theta-b (Q_{b}) respectively are not measured. Existing needle visualization enhancement features assume this value via a default parameter or require the user to estimate the needle angle and input it via the user interface. View ‘c’ 1103 shows how the beam can be optimally steered when the angle of the needle theta-c (Q_{c}) is known via the kinematics of the robot. Needle visualization enhancement done via post-processing of the image (i.e. not by steering the ultrasound beam) relies on similar information of the needle pose. Needle visualization enhancement can then be presented to the user in several ways on the user interface. An example of this is enhancing the contrast of the image in the known region of the instrument, or by adding color overlays.

An alternative embodiment of needle enhancement is by performing machine learning segmentation on the region of the image where the needle is expected to be visualized. This could be used in conjunction with the beam steering embodiment above but could also be performed independently. By focusing the segmentation algorithm on a smaller region of the image, the performance and speed of the needle identification can be improved.

Inverse kinematics (IK) based constrained backdriving may be described herein. Unlike conventional unconstrained backdriving, where the user has the burden of controlling all degrees-of-freedom (Doff) of a given robot's motion, this inverse kinematics (IK) based constrained backdriving lets the user only focus on the part they need to control and assists the rest of the motion kinematically in a human-in-the-loop fashion. Given the same framework, it may be very flexible and easy to implement a range of constrained backdriving modes to meet specific desires, and/or migrate to different robot designs. The user experience mainly depends on the motion constraint and user touchpoint selections for a given robot.

The common framework of IK-based constrained backdriving, and the design 3 specific constrained backdriving modes, namely a) RCM (remote center of motion) backdriving; b) PnP (placement and pointing) backdriving, and c) PnT (placement and tracking) backdriving are described herein. Each of the 3 constrained backdriving modes has a correspondent automated motion mode for similar targeting needs: a) RCM backdriving to T2T; b) PnP backdriving to D2R; and c) PnT backdriving to dynamic targeting (such as automatically tracking anatomy that has been identified by segmenting pixels in the ultrasound image). The user touchpoints of different backdriving modes may or may not overlap. On the other hand, for a backdriving mode, the adaptation to a 3-DoF robot vs. 5-DoF robots.

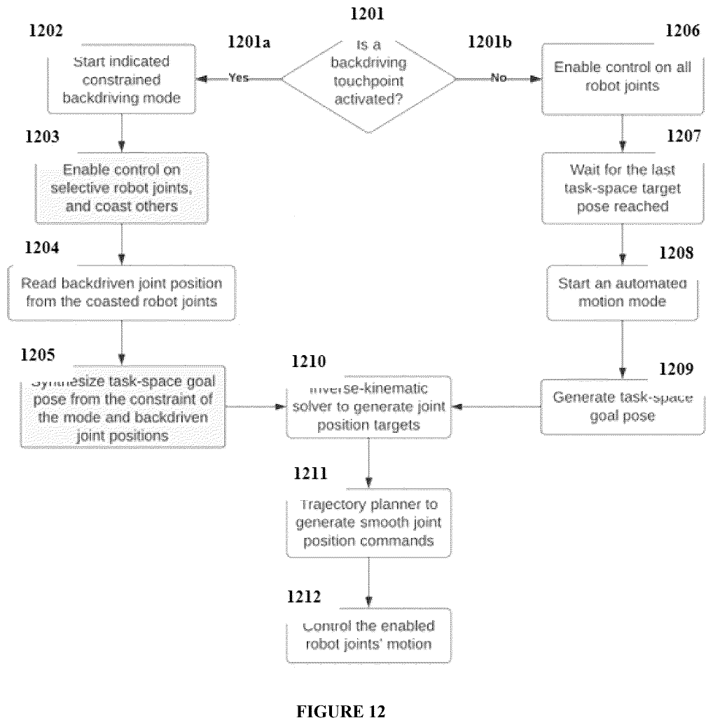

FIG. 12 is an example flowchart of the constrained backdriving framework. FIG. 12 illustrates not only the common flow of constrained backdriving, but also switching among all modes of robot operation, where unconstrained backdriving may be treated as a special case of 0-constrained backdriving. FIG. 12 details the steps that occur both when a backdriving touchpoint is and is not activated 1201.

When a backdriving touch point is activated 1201a, the system may start indicating constrained backdriving mode 1202. The control on selective robot joints may then be enabled and coast others 1203. The backdriven joint position from the coasted robot joints may then be read 1204. The task-space goal pose may then be synthesized from the constraint of the mode and backdriven joint positions 1205. An inverse-kinematic solver may then be used to generate joint position targets 1210.

When a backdriving touch point is not activated 1201b, control on all robot joints may be activated 1206. The system may then wait for the last task-space target posed to be reached 1207. An automated motion mode may then be started 1208. A task-space goal pose may then be generated 1209. An inverse-kinematic solver may then be used to generate joint position targets 1210.

In both backdriving framework 1201a and 1201b, the trajectory planner may then generate smooth joint position commands 1211. The enabled robot joints' motion may then be controlled 1212.

For different constrained backdriving modes, there are only two highlighted areas in FIG. 12, for example, selective robot joints under control 1203 and synthesizing task-space goal pose 1205, need to be devised based on the targeting requirement and robot design. The details on the 3 modes are described herein.

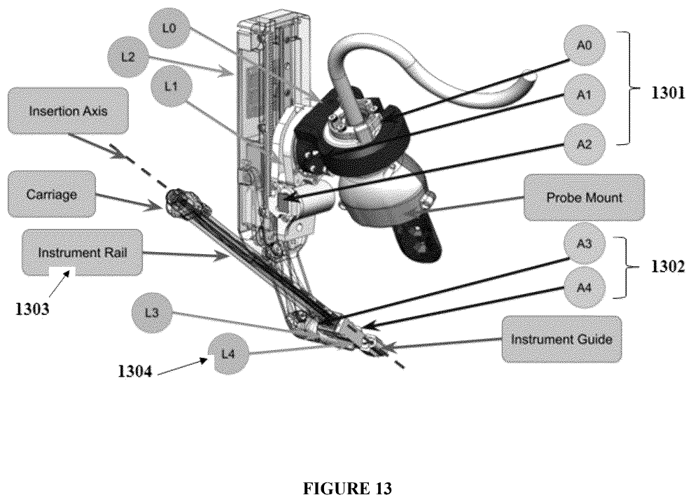

A first mode may be RCM backdriving. RCM backdriving is where a user needs to maintain a constant location of the skin-entry site but wants to adjust the needle angle for pointing to a new target. Therefore, control may be enabled on proximal joints to maintain (or converge to) the constant task-space end effector position while coasting the distal rotational joint(s) for the user to adjust the orientation. Specifically, for the exemplary 5-DoF robot in FIG. 13 (described in further detail below), A0˜A2 1301 are under control for the 3-DoF task-space end effector position, and A3˜A4 1302 under coast; the A3/A4's backdriven joint angles are transformed to the constrained task-space location to synchronize the full 5-DoF task-space goal pose to be fed into its IK solver to compute the goal pose of A0˜A2.

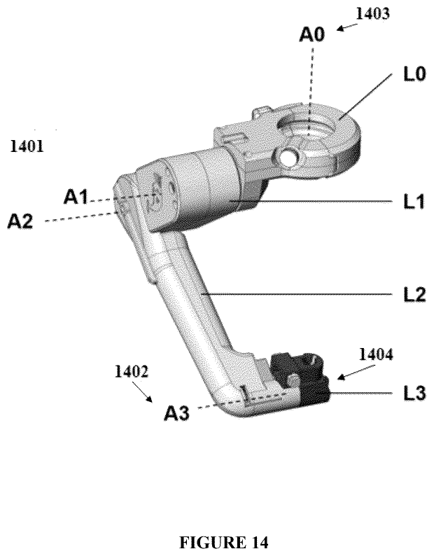

For the exemplary-3-DoF robot in FIG. 14 (described in further detail below), it's A1˜A2 1401 under control for the 2-DoF task-space location, and A3 1402 under coast; a synthesized 3-DoF task-space goal pose is provided to its IK solver to compute the pose of A1˜A2 1401. In general, the task space end effector position that is being kept constant is referred to the RCM. This point may coincide with the tip of the needle guide, the location along the instrument where it is inserted into the skin, the location in space associated with anatomy of interest, or the like.

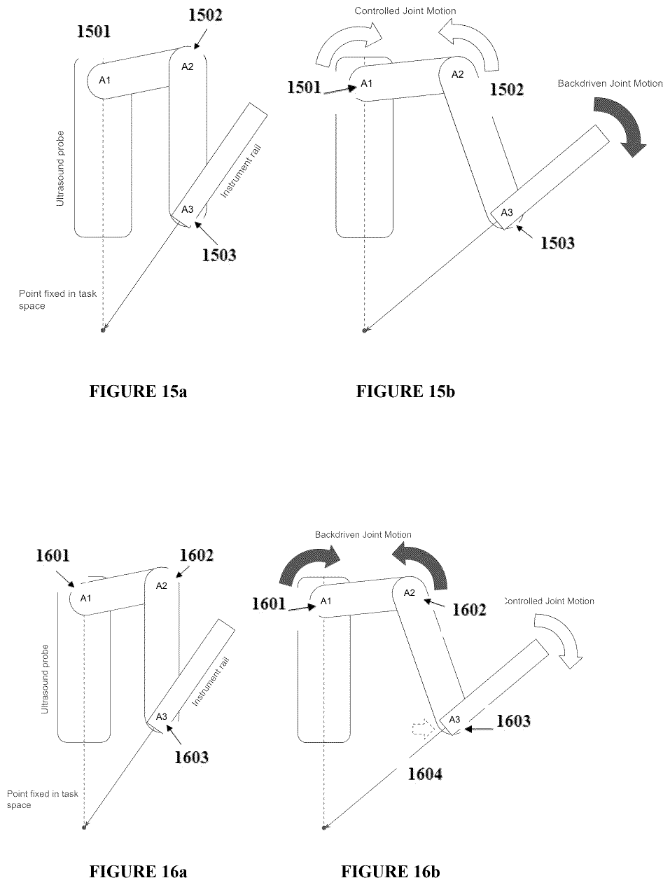

FIG. 15 illustrates an example of RCM backdriving. Here the RCM is a point fixed in task space defined underneath the ultrasound probe. The user backdrives the A3 1503 joint as shown in FIG. 15b, and joints A1 1501 and A2 1502 are controlled to extend the arm such that the instrument is still pointing at the RCM with the newly defined angle.

A second mode may be PnP backdriving. PnP backdriving may be complementary to RCM backdriving, because the user backdrives the proximal joints that define the position of the end effector, while the distal joint is controlled to ensure that the instrument is consistently pointing at the target of interest. Consequently, control may be enabled on distal rotational joint(s) for pointing while coasting the proximal joints to let the user backdrive the task-space location. Specifically, for the exemplary 5-DoF robot, A0˜A2 are under coast, and A3˜A4 are under control; the 2-DoF orientation is derived from the target and the robot end-effector positions to complete the 5-DoF task-space goal pose for its IK solver. For the exemplary 3-DoF robot, A1˜A2 are under coast, and A3 under control; the 1-DoF orientation is derived to synthesize the 3-DoF task-space goal pose for its IK solver. In general, the location in space that the robot end effector is pointing to can be arbitrarily defined.

FIG. 16 illustrates an example of PnP backdriving. Here the user interaction is to drag the end effector from a location in FIG. 16a to a new location denoted by the dashed arrow 1604 in FIG. 16b. Joints A1 1601 and A2 1602 are backdriven by way of repositioning of the end effector. Joint A3 1603 is actively controlled to point the instrument at a fixed point in task space.

A third mode may be PnT backdriving. PnT backdriving is almost identical to PnP backdriving, except that it may be used to point to a dynamic target. Therefore, the backdriving portion is identical to PnP backdriving, w/ the target being updated in real-time by another module, such as the dynamic targeting described above.

There are many technical advantages to backdriving. Backdriving may be simple, cheap, robust and performant.

Comparing to alternative solutions in collaborative robotics (cobot), backdriving avoids the need of torque sensors and/or the complexity of admittance control or direct torque control. This is particularly important for a hand-held robot where additional torque sensors can be heavy. IK and joint-control enabling selection may fully decouple the robot in joint-space, so the same decoupled performant and safety architecture of automated motion modes apply to constrained backdriving, with hard and fast real-time loop on joint control and complex human-in-the-loop portion running just fast enough comparing to human reaction bandwidth.

Since the robot control for both backdriving and automated motion modes are purely kinematics based, they are under the same framework as described herein. The mode-switching and workflow support may be straightforward and robust.

Backdriving is intuitive and effective for user interaction. Physical interaction and haptic/tactile feedback may be available without adding any additional fine targeting burden to the user (the best of both unconstrained backdriving and fully automated motion). Backdriving may minimize the interruptions caused by switching the operations back and forth between the physical robot and the GUI input. A user may opt to largely use GUI for visual feedback only without needing to take their hands off the robot/probe or patient. Backdriving may relieve GUI's 2D constraint in designing user interaction. For example, D2R is for in-plane operations only, while PnP backdriving may be used at any orientation.

Backdriving may be compatible with other enhancements. For example, it may easily add joint-space impedance control, such as gravity/friction/backlash compensation etc., to further enhance the backdriving experience. In another example, it may easily add more modes to enhance user experience in a new area, e.g. PnT backdriving to enhance dynamic targeting or PnP backdriving for out of plane operation.

Inverse kinematics may be used to constrain robot backdriving. The 3 specific constrained-backdriving modes for different robot designs may be used to preserve both the full fine targeting capabilities of automated motion and the backdriving user interaction w/haptic/tactile feedback. Transition among fully automated motion control and (constrained or unconstrained) backdriving modes via IK and trajectory planning may be utilized. Enabling “always hands-on” operation w/full sensory feedback to a user (visual from GUI and haptic/tactile from direct interaction w/robot/probe and/or patient) may be utilized.

FIG. 13 is an example of a 5-DoF robot design. A0-A4 denote actuated joints of the robot. These actuated joints may be linear or rotational. L0-L4 denote the links of the robot. The touchpoint for RCM backdriving may be at the proximal end of Instrument Rail 1303, while that of unconstrained backdriving, PnP backdriving, and PnT backdriving may all be the same, in the vicinity of L4 1304.

FIG. 14 is an example of a 3-DoF robot design. A1-A3 denote actuated joints of the robot. These actuated joints may be linear or rotational. L1-L3 denote the links of the robot. As illustrated A0 1403 is not active and thus A1˜A3 are the actual 3-DoF. Additionally, an Instrument Rail may be included (not shown). The touchpoint for RCM backdriving may be at the proximal end of Instrument Rail, while that of unconstrained backdriving, PnP backdriving, and PnT backdriving may all be the same, in the vicinity of L3 1404.

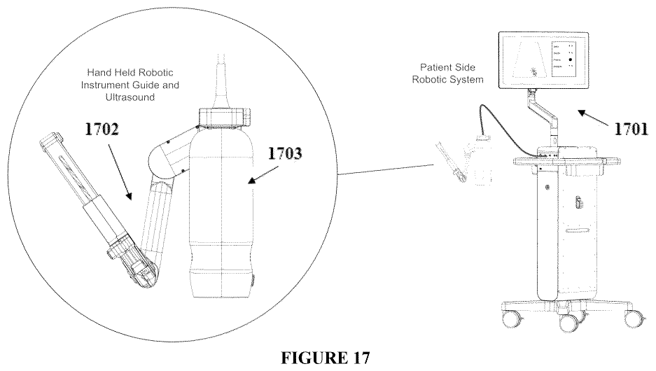

FIG. 17 is an example of a patient side robotic system for percutaneous instrument intervention. The two major subsystems are a patient side cart 1701 and a hand-held robotic instrument guide 1702 with ultrasound probe 1703. The computer, power supply, usb communication components and display are part of the patient side cart subsystem 1701. The hand-held robotic subsystem plugs into the patient side cart 1701 via a cable which facilitates digital communication and power. The handheld robotic subsystem is comprised of an ultrasound probe 1703, robotic positioning arm 1702, and instrument guide (not shown).

The user holds the ultrasound (with robot attached) in one hand and manipulates the robot end effector/instrument and graphical user interface with the other. This paradigm may allow the robot to target objects seen in the ultrasound image by tapping the target—or other controls—on the display. Ultrasound settings are also available to the user. When the user is satisfied with the targeting configuration, they are able to insert the instrument through the guide to the target. Graphics are displayed on the screen that denote the path that the instrument will take, and the location of the instrument tip as the instrument is inserted into the patient.

1. A system comprising:
a robotic system comprising:
a robotic arm, wherein the robotic arm includes a robotic manipulator and an instrument guide couple to the robotic manipulator; and
a medical imaging device;
a system user interface, wherein the system user interface displays a plurality of medical images and robotic system controls;
a remote user interface provided to at least one remote user, wherein the remote user interface includes at least one of: at least one element of the system user interface and at least one image from the medical imaging device; and
two-way communication capabilities between the at least one remote user and a robot operator, wherein the two-way communication capabilities includes at least one of: an audio communication and a video communication.

2. The system of claim 1, wherein the robotic system facilitates percutaneous medical interventions.

3. The system of claim 1, wherein the medical imaging device is an ultrasound probe.

4. The system of claim 1, wherein the at least one remote user interacts with elements of the system user interface remotely.

5. The system of claim 1, wherein the remote user is provided a portal with robotic system telemetry data.

6. The system of claim 1, wherein the remote user modifies a robotic system software.

7. The system of claim 1, wherein the remote user interface is a duplicate of the system user interface.

8. The system of claim 1, wherein the at least one remote user utilizes telestration to provide guidance to the robot operator.

9. The system of claim 1, wherein the system user interface utilizes a graphical overlay.

10. A non-transitory computer readable storage media comprising instructions, the instructions executable by a processor to perform a method, the method comprising:
receiving, from an imaging device couple to a robotic manipulator, a plurality of medical images;
displaying the received plurality of medical images and robotic system controls on a system user interface
displaying on a remote user interface at least one of:
at least one element of the system user interface and
at least one image from the medical imaging device
wherein the remote user interface is provided to at least one remote user; and
enabling two-way communication capabilities between the at least one remote user and a robot operator, wherein the two-way communication capabilities includes at least one of: an audio communication and a video communication.

11. The non-transitory computer readable storage media of claim 10, wherein the robotic system facilitates percutaneous medical interventions.

12. The non-transitory computer readable storage media of claim 10, wherein the medical imaging device is an ultrasound probe.

13. The non-transitory computer readable storage media of claim 10, wherein the at least one remote user interacts with elements of the system user interface remotely.

14. The non-transitory computer readable storage media of claim 10, wherein the remote user is provided a portal with robotic system telemetry data.

15. The non-transitory computer readable storage media of claim 10, wherein the remote user modifies a robotic system software.

16. The non-transitory computer readable storage media of claim 10, wherein the remote user interface is a duplicate of the system user interface.

17. The non-transitory computer readable storage media of claim 10, wherein the at least one remote user utilizes telestration to provide guidance to the robot operator.

18. The non-transitory computer readable storage media of claim 10, wherein the system user interface utilizes a graphical overlay.
